(19) United States
(12) Patent Application Publication
(10) Pub. No.: US 2023/0028788 A1
(43) Pub. Date: Jan. 26, 2023

(54) T CELL PERFORMANCE ASSAY AS A PROGNOSTIC FACTOR FOR CLINICAL OUTCOME

(71) Applicant: Baylor College of Medicine, Houston, TX (US)

(72) Inventors: Wingchi Leung, Houston, TX (US); Premal Lulla, Houston, TX (US); Spyridoula Vasileiou, Houston, TX (US); Ann Marie Leen, Houston, TX (US); Juan F. Vera Valdes, Houston, TX (US)

(21) Appl. No.: 17/757,881

(22) PCT Filed: Dec. 18, 2020
(86) PCT No.: PCT/US2020/065968

§ 371 Date: Jun. 22, 2022

Related U.S. Application Data
(60) Provisional application No. 62/952,718, filed on Dec. 23, 2019.

(51) Int. Cl. G01N 33/574; G01N 33/50; C12N 5/078

(52) U.S. Cl. CPC G01N 33/574; G01N 33/505; C12N 5/0634; G01N 2800/7028

(57) ABSTRACT

The present disclosure provides, inter alia, compositions and methods for harnessing the immune system (e.g., T cells) as a detection tool to diagnose and predict cancer patient treatment outcomes and for monitoring the persistence of functional, non-genetically modified, T cell therapies in vivo after administration to a subject.

This application claims priority to U.S. Provisional Patent Application Ser. No. 62/952,718, filed Dec. 23, 2019, which is incorporated by reference herein in its entirety.

### TECHNICAL FIELD

Embodiments of the disclosure concern at least the fields of cell biology, molecular biology, immunology, and medicine.

### BACKGROUND

Conventional cancer diagnostics is a stepwise process, often initiated upon patients seeking medical care due to symptoms. Subsequently, blood tests, imaging scans, and biopsies (for morphological, immunophenotypical and and/or cytogenetic studies) are performed to identify, confirm, characterize and stage the cancer.

Although this is the current state of the art, this strategy is limited to patients with evident symptoms. However, in those who are asymptomatic and/or have small disease detection may be missed due to the limited sensitivity of conventional diagnostic approaches including imaging. Biopsies are invasive procedures which are only feasible for safely accessible lesions. Furthermore, as only limited material is obtained, the attained biopsy sample may not represent the actual tumor lesion, or other metastatic lesions at distant sites within the patient. Lastly, these conventional tests do not characterize the specific immune response to the tumor, which influences cancer progression and clinical outcome. This disclosure addresses this and other needs.

### SUMMARY OF CERTAIN EMBODIMENTS

Embodiments of the present disclosure satisfy a long-felt need in the art of cancer diagnostics/prognostics by providing inter alia novel methods in which the immune system (specifically T cells) is harnessed as a detection tool to diagnose and prognosticate cancer patients. In some embodiments, the present disclosure provides an in vitro method for detecting the spectrum, quality and quantity of tumor-directed T cells in the patients.

In some embodiments, peripheral blood mononuclear cells are obtained and stimulated (directly or with antigen presenting cells such as dendritic cells) using synthetic peptides spanning selected tumor antigen sequences. The immune response mounted by T cells (as determined by antigen-stimulated cytokine production, e.g., interferon-g secretion) is used as a surrogate marker for diagnosis and prognosis. As T cells arise in response to disease burden that is below the threshold of conventional detection methods, this information is used for the early diagnosis and treatment monitoring to predict outcomes in cancer patients.

The present disclosure provides, inter alia, compositions and methods for harnessing the immune system (e.g., T cells) as a detection tool to diagnose and predict cancer patient treatment outcomes. Embodiments of the disclosure encompass methods of diagnosing the presence of a tumor in a subject comprising: (a) culturing peripheral blood mononuclear cells (PBMCs) obtained from the subject in culture with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject; (b) contacting one or more of the T cells that have undergone expansion according to step (a) with (i) at least one of the pepmix libraries included in the said plurality of pepmix libraries, or (ii) at least one of the plurality of overlapping peptides contained in one of said pepmix libraries; (c) measuring whether the contacting in (b) results in an immune response elicited by the one or more T cells; and (d) diagnosing the subject as having the tumor if the contacting in (b) does result in an immune response as measured in (c).

Particular embodiments include methods of diagnosing the presence of a tumor in a subject comprising: (a) culturing memory T cells obtained from the subject with dendritic cells that have been contacted with a plurality of pepmix libraries, each library comprising a plurality of overlapping peptides that combine to cover a known tumor associated antigen (TAA); wherein culturing results in stimulating the expansion of one or more memory T cells obtained from the subject; (b) contacting one or more of the memory T cells that have undergone expansion according to step (a) with (i) at least one of the said pepmix libraries included in the plurality of pepmix libraries, or (ii) at least one of the plurality of overlapping peptides contained in one of said pepmix libraries; (c) measuring whether the contacting in (b) results in an immune response elicited by the one or more T cells; and (d) diagnosing the subject as having the tumor if the contacting in (b) does result in an immune response as measured in (c). In any of the methods encompassed herein, the T cells are isolated from PBMCs obtained from the subject. Any culturing steps of any methods disclosed herein may cover a period of at least or no more than or exactly 7-10 days. In particular embodiments, the cells are in the presence of at least 2, 3, 4, 5, 6, 7, 8, 9, 10, 11, 12, 13, 14, 15, 16, 17, 18, 19, 20, or more different pepmixes, each pepmix comprising a series of overlapping peptides that span part of or the entire sequence of an antigen. As one example, at least 2, 3, 4, 5, 6, 7, 8, 9, 10, 11, 12, 13, 14, 15, 16, 17, 18, 19, 20 or more different antigens are covered by the plurality of pepmixes. In specific cases, the pepmix comprises 15 mer peptides. The pepmix may comprise 7 mer, 8 mer, 9 mer, 10 mer, 11 mer, 12 mer, 13 mer, 14 mer, or 15 mer or greater peptides. In any case, the peptides in the pepmix that span the antigen may overlap in sequence by 7, 8, 9, 10, 11, 12, 13, or 14 or more amino acids.

In one embodiment, there is a method of predicting whether a cancer patient is likely to respond to multi TAA T-cell therapy, comprising: performing steps (a) through (c) set forth above; quantifying the magnitude of the immune response from step (c); and comparing the magnitude of the patient's immune response with a reference immune response selected from a response generated in a similar method using one or more reference samples from subjects with known treatment outcomes (i.e. known to have relapsed, or not) or historical response values from one or more reference samples from subjects with known treatment outcomes (i.e. known to have relapsed, or not); wherein (A) if the immune response from step (c) is comparable to the reference immune response of a patient known to have relapsed, the patient is determined to have a high likelihood of relapsing; and (B) if the immune response from step (c) is comparable to the reference immune response of a patient known to not have relapsed, the patient is determined to have a low likelihood of relapsing. In specific cases, if the patient is determined to have a high likelihood of relapsing, the patient is treated with chemotherapy, immunotherapy, radiation therapy, resection surgery, transplant (solid tissue or stem cell) or a combination thereof. If the patient is determined to have a low likelihood of relapsing, steps (a) through (c) set forth above may be repeated one or more additional times over time to monitor whether any temporal changes in the immune responses elicited by the one or more T cells is detectable. If a temporal change in the immune responses elicited by the one or more T cells is detectable, the magnitude of the change is quantified and compared one or more time to the magnitude of the immune response of a reference immune response as set forth in methods herein. If the patient is determined to have a high likelihood of relapsing after one or more time of comparing the magnitude of patient's immune response with a reference immune response, the patient is treated with chemotherapy, immunotherapy, radiation therapy, resection surgery, transplant (solid tissue or stem cell) or a combination thereof. In embodiments, the same method is performed except that step (a) comprises culturing memory T cells obtained from the subject with dendritic cells that have been contacted with a plurality of pepmix libraries, each library comprising a plurality of overlapping peptides that combine to cover a known tumor associated antigen (TAA); wherein culturing results in stimulating the expansion of one or more memory T cells obtained from the subject.

In a particular embodiment, there is a method for detecting antigen loss comprising: (a) culturing peripheral blood mononuclear cells (PBMCs) obtained from the subject at a first time point (“baseline”) with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject; (b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries; (c) measuring and quantifying the magnitude of any immune response elicited by the one or more T cells in response to the contacting in (b); (d) repeating steps (a) through (c) one or more additional times with PBMCs obtained from the subject at one or more subsequent time points; (e) comparing the magnitude of the immune responses from the first and one or more of each subsequent quantifying step; and (f) identifying any immune responses that change in magnitude over time; wherein a change in an immune response elicited by a particular pepmix covering a known TAA, or by a peptide comprised in such a pepmix, indicates a change in the expression of that TAA by a tumor in the patient. In some cases, the first time point is before the patient receives a treatment for the cancer and each subsequent time point is after the patient receives the treatment. In certain cases, the first time point and a first subsequent time point are separated by a period ranging from one week to one year, one week to six months or one week to one month. The method may encompass 2, 3, 4, 5, 6, 7, 8, 9, 10, 11, 12, 13, 14, 15, 16, 17, 18, 19, or 20 subsequent time points. The treatment may be of any kind, including one selected from a chemotherapy, immunotherapy, radiation therapy, resection surgery, solid tissue transplant, a stem cell transplant, or a combination thereof. The treatment may be an autologous or allogenic antigen specific T cell therapy.

In one embodiment, there is a method of predicting whether a patient is likely to relapse after a remission, comprising: (a) culturing peripheral blood mononuclear cells (PBMCs) in culture with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject; (b) contacting one or more of the T cells that have undergone expansion according to step (a) with (i) at least one of the pepmix libraries included in the said plurality of pepmix libraries, or (ii) at least one of the plurality of overlapping peptides contained in one of said pepmix libraries; (c) measuring whether the contacting in (b) results in an immune response elicited by the one or more T cells; quantifying the magnitude of the immune response from step (c); and comparing the magnitude of the patient's immune response with a reference immune response selected from a response generated in a similar method using one or more reference samples from subjects with known treatment outcomes (i.e. known to have relapsed, or not) or historical response values from one or more reference samples from subjects with known treatment outcomes (i.e. known to have relapsed, or not); wherein (A) if the immune response from step (c) is comparable to the reference immune response of a patient known to have relapsed, the patient is determined to have a high likelihood of relapsing; and (B) if the immune response from step (c) is comparable to the reference immune response of a patient known to not have relapsed, the patient is determined to have a low likelihood of relapsing. If the patient is determined to have a high likelihood of relapsing, the patient is treated with chemotherapy, immunotherapy, radiation therapy, resection surgery, transplant (solid tissue or stem cell) or a combination thereof. If the patient is determined to have a low likelihood of relapsing, steps (a) through (c) set forth above in this paragraph are repeated one or more additional times over time to monitor whether any temporal changes in the immune responses elicited by the one or more T cells is detectable. If a temporal change in the immune responses elicited by the one or more T cells is detectable, the magnitude of the change is quantified and compared one or more time to the magnitude of the immune response of a reference immune response as set forth in methods herein. If the patient is determined to have a high likelihood of relapsing after one or more time of comparing the magnitude of patient's immune response with a reference immune response, the patient is treated with chemotherapy, immunotherapy, radiation therapy, resection surgery, transplant (solid tissue or stem cell) or a combination thereof. In embodiments, the same method is performed except that step (a) is performed by culturing memory T cells obtained from the subject with dendritic cells that have been contacted with a plurality of pepmix libraries, each library comprising a plurality of overlapping peptides that combine to cover a known tumor associated antigen (TAA); wherein culturing results in stimulating the expansion of one or more memory T cells obtained from the subject.

In a particular embodiment there is a method for detecting changes in the TAA expression profile of a tumor over time comprising: (a) culturing peripheral blood mononuclear cells (PBMCs) obtained from the subject at a first time point (“baseline”) with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject; (b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries; (c) measuring and quantifying the magnitude of any immune response elicited by the one or more T cells in response to the contacting in (b); repeating steps (a) through (c) one or more additional times with PBMCs obtained from the subject at one or more subsequent time points; comparing the magnitude of the immune responses from the first and one or more of each subsequent quantifying step; and identifying any immune responses that change in magnitude over time; wherein a change in an immune response elicited by a particular pepmix covering a known TAA, or by a peptide comprised in such a pepmix, indicates a change in the expression of that TAA by a tumor in the patient. In certain embodiments, the first time point is before the patient receives a treatment for the cancer and each subsequent time point is after the patient receives the treatment. In some embodiments, the first time point and a first subsequent time point are separated by a period ranging from one week to one year, one week to six months or one week to one month. Some methods may comprise at least 2, 3, 4, 5, 6, 7, 8, 9, 10, 11, 12, 13, 14, 15, 16, 17, 18, 19, or 20 subsequent time points. The treatment may be selected from a chemotherapy, immunotherapy, radiation therapy, resection surgery, solid tissue transplant, a stem cell transplant, or a combination thereof, including an autologous or allogenic antigen specific T cell therapy. In embodiments, the same method is performed except that step (a) comprises culturing memory T cells obtained from the subject with dendritic cells that have been contacted with a plurality of pepmix libraries, each library comprising a plurality of overlapping peptides that combine to cover a known tumor associated antigen (TAA); wherein culturing results in stimulating the expansion of one or more memory T cells obtained from the subject.

In certain embodiments, there is a method for monitoring the in vivo persistence of antigen specific T cells that have not been genetically modified and that have been administered to a subject, the method comprising (a) culturing peripheral blood mononuclear cells (PBMCs) obtained from the subject at a first time point (“baseline”) with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject; (b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries; (c) measuring whether the contacting in (b) results in an immune response elicited by the one or more T cells; wherein the measuring optionally comprises quantifying the magnitude of any immune response elicited by the one or more T cells in response to the contacting in (b); repeating steps (a) through (c) one or more additional times with PBMCs obtained from the subject at one or more subsequent time points; comparing the baseline immune response with one or more subsequent immune responses, wherein optionally the comparing comprises comparing the magnitude of the immune responses, wherein the detection of an immune response from one or more of the subsequent time points that is greater than or equal to the immune response generated by T cells obtained from the patient at the baseline time indicates that the antigen specific T cells still persist; and wherein the detection of an immune response from one or more of the subsequent time points that is less than the immune response generated by T cells obtained from the patient at the baseline time indicates that the antigen specific T cells no longer persist. In embodiments, the same method is performed except that step (a) comprises culturing memory T cells obtained from the subject with dendritic cells that have been contacted with a plurality of pepmix libraries, each library comprising a plurality of overlapping peptides that combine to cover a known tumor associated antigen (TAA); wherein culturing results in stimulating the expansion of one or more memory T cells obtained from the subject.

In specific embodiments, there is a method of diagnosing the presence of a tumor in a subject comprising: (a) culturing peripheral blood mononuclear cells (PBMCs) obtained from the subject in culture with a plurality of pepmix libraries, each library comprising a plurality of overlapping peptides that combine to cover a known tumor specific antigen (TSA); wherein culturing results in stimulating the expansion of one or more T cells contained within the PBMCs obtained from the subject; (b) contacting one or more of the T cells that have undergone expansion according to step (a) with (i) at least one of the pepmix libraries included in the said plurality of pepmix libraries, or (ii) at least one of the plurality of overlapping peptides contained in one of said pepmix libraries; (c) measuring whether the contacting in (b) results in an immune response elicited by the one or more T cells; and diagnosing the subject as having the tumor if the contacting in (b) does result in an immune response as measured in (c). In embodiments, the same method is performed except that step (a) comprises culturing memory T cells obtained from the subject with dendritic cells that have been contacted with a plurality of pepmix libraries, each library comprising a plurality of overlapping peptides that combine to cover a known tumor associated antigen (TAA); wherein culturing results in stimulating the expansion of one or more memory T cells obtained from the subject.

In some embodiments, there is a method of diagnosing the presence of a tumor in a subject comprising: (a) culturing memory T cells obtained from the subject with dendritic cells that have been contacted with a plurality of pepmix libraries, each library comprising a plurality of overlapping peptides that combine to cover a known tumor specific antigen (TSA); wherein culturing results in stimulating the expansion of one or more memory T cells obtained from the subject; (b) contacting one or more of the memory T cells that have undergone expansion according to step (a) with (i) at least one of the said pepmix libraries included in the plurality of pepmix libraries, or (ii) at least one of the plurality of overlapping peptides contained in one of said pepmix libraries; measuring whether the contacting in (b) results in an immune response elicited by the one or more T cells; and diagnosing the subject as having the tumor if the contacting in (b) does result in an immune response as measured in (c). In specific cases, the T cells are isolated from PBMCs obtained from the subject. The culturing step (a) may cover a period of 7-10 days. In specific embodiments, the cells are in the presence of at least 2, 3, 4, 5, 6, 7, 8, 9, 10, 11, 12, 13, 14, 15, 16, 17, 18, 19, 20, or more different pepmixes, each pepmix comprising a series of overlapping peptides that span part of or the entire sequence of an antigen; the peptides in the pepmix that span the antigen may overlap in sequence by 11 amino acids.

In one embodiment, there is a method of predicting whether a cancer patient is likely to respond to multi TSA T-cell therapy, comprising: (a) culturing peripheral blood mononuclear cells (PBMCs) in culture with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject; (b) contacting one or more of the T cells that have undergone expansion according to step (a) with (i) at least one of the pepmix libraries included in the said plurality of pepmix libraries, or (ii) at least one of the plurality of overlapping peptides contained in one of said pepmix libraries; (c) measuring whether the contacting in (b) results in an immune response elicited by the one or more T cells; quantifying the magnitude of the immune response from step (c); and comparing the magnitude of the patient's immune response with a reference immune response selected from a response generated in a similar method using one or more reference samples from subjects with known treatment outcomes (i.e. known to have relapsed, or not) or historical response values from one or more reference samples from subjects with known treatment outcomes (i.e. known to have relapsed, or not); wherein (A) if the immune response from step (c) is comparable to the reference immune response of a patient known to have relapsed, the patient is determined to have a high likelihood of relapsing; and (B) if the immune response from step (c) is comparable to the reference immune response of a patient known to not have relapsed, the patient is determined to have a low likelihood of relapsing. If the patient is determined to have a high likelihood of relapsing, the patient is treated with chemotherapy, immunotherapy, radiation therapy, resection surgery, transplant (solid tissue or stem cell) or a combination thereof. If the patient is determined to have a low likelihood of relapsing, steps (a) through (c) set forth above are repeated one or more additional times over time to monitor whether any temporal changes in the immune responses elicited by the one or more T cells is detectable. If a temporal change in the immune responses elicited by the one or more T cells is detectable, the magnitude of the change is quantified and compared one or more time to the magnitude of the immune response of a reference immune response as set forth in methods encompassed herein. If the patient is determined to have a high likelihood of relapsing after one or more time of comparing the magnitude of patient's immune response with a reference immune response, the patient is treated with chemotherapy, immunotherapy, radiation therapy, resection surgery, transplant (solid tissue or stem cell) or a combination thereof.

In certain embodiments, there is a method for detecting antigen loss comprising: (a) culturing peripheral blood mononuclear cells (PBMCs) obtained from the subject at a first time point (“baseline”) with a plurality of pepmix libraries, each library comprising a plurality of overlapping peptides that combine to cover a known tumor specific antigen (TSA); wherein culturing results in stimulating the expansion of one or more T cells contained within the PBMCs obtained from the subject; (b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries; (c) measuring and quantifying the magnitude of any immune response elicited by the one or more T cells in response to the contacting in (b); repeating steps (a) through (c) one or more additional times with PBMCs obtained from the subject at one or more subsequent time points; comparing the magnitude of the immune responses from the first and one or more of each subsequent quantifying step; and identifying any immune responses that change in magnitude over time; wherein a change in an immune response elicited by a particular pepmix covering a known TSA, or by a peptide comprised in such a pepmix, indicates a change in the expression of that TSA by a tumor in the patient. In some cases, the first time point is before the patient receives a treatment for the cancer and each subsequent time point is after the patient receives the treatment. In some cases, the first time point and a first subsequent time point are separated by a period ranging from one week to one year, one week to six months or one week to one month. In specific aspects, the method comprises 2, 3, 4, 5, 6, 7, 8, 9, 10, 11, 12, 13, 14, 15, 16, 17, 18, 19, or 20 subsequent time points. The treatment may be selected from a chemotherapy, immunotherapy, radiation therapy, resection surgery, solid tissue transplant, a stem cell transplant, or a combination thereof. The treatment may be an autologous or allogenic antigen specific T cell therapy.

In one embodiment, there is a method of predicting whether a patient is likely to relapse after a remission, comprising: (a) culturing peripheral blood mononuclear cells (PBMCs) in culture with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject; (b) contacting one or more of the T cells that have undergone expansion according to step (a) with (i) at least one of the pepmix libraries included in the said plurality of pepmix libraries, or (ii) at least one of the plurality of overlapping peptides contained in one of said pepmix libraries; (c) measuring whether the contacting in (b) results in an immune response elicited by the one or more T cells; quantifying the magnitude of the immune response from step (c); and comparing the magnitude of the patient's immune response with a reference immune response selected from a response generated in a similar method using one or more reference samples from subjects with known treatment outcomes (i.e. known to have relapsed, or not) or historical response values from one or more reference samples from subjects with known treatment outcomes (i.e. known to have relapsed, or not); wherein (A) if the immune response from step (c) is comparable to the reference immune response of a patient known to have relapsed, the patient is determined to have a high likelihood of relapsing; and (B) if the immune response from step (c) is comparable to the reference immune response of a patient known to not have relapsed, the patient is determined to have a low likelihood of relapsing. If the patient is determined to have a high likelihood of relapsing, the patient is treated with chemotherapy, immunotherapy, radiation therapy, resection surgery, transplant (solid tissue or stem cell) or a combination thereof. If the patient is determined to have a low likelihood of relapsing, steps (a) through (c) set forth above are repeated one or more additional times over time to monitor whether any temporal changes in the immune responses elicited by the one or more T cells is detectable. In specific embodiments, if a temporal change in the immune responses elicited by the one or more T cells is detectable, the magnitude of the change is quantified and compared one or more time to the magnitude of the immune response of a reference immune response as set forth in methods encompassed herein. If the patient is determined to have a high likelihood of relapsing after one or more time of comparing the magnitude of patient's immune response with a reference immune response, the patient is treated with chemotherapy, immunotherapy, radiation therapy, resection surgery, transplant (solid tissue or stem cell) or a combination thereof.

In some embodiments, there is a method for detecting changes in the TSA expression profile of a tumor over time comprising: (a) culturing peripheral blood mononuclear cells (PBMCs) obtained from the subject at a first time point (“baseline”) with a plurality of pepmix libraries, each library comprising a plurality of overlapping peptides that combine to cover a known tumor specific antigen (TSA); wherein culturing results in stimulating the expansion of one or more T cells contained within the PBMCs obtained from the subject; (b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries; (c) measuring and quantifying the magnitude of any immune response elicited by the one or more T cells in response to the contacting in (b); repeating steps (a) through (c) one or more additional times with PBMCs obtained from the subject at one or more subsequent time points; comparing the magnitude of the immune responses from the first and one or more of each subsequent quantifying step; and identifying any immune responses that change in magnitude over time; wherein a change in an immune response elicited by a particular pepmix covering a known TSA, or by a peptide comprised in such a pepmix, indicates a change in the expression of that TSA by a tumor in the patient. The first time point may be before the patient receives a treatment for the cancer and each subsequent time point is after the patient receives the treatment. The first time point and a first subsequent time point may be separated by a period ranging from one week to one year, one week to six months or one week to one month. The method may comprise 2, 3, 4, 5, 6, 7, 8, 9, 10, 11, 12, 13, 14, 15, 16, 17, 18, 19, or 20 subsequent time points. In some cases, the treatment is selected from a chemotherapy, immunotherapy, radiation therapy, resection surgery, solid tissue transplant, a stem cell transplant, or a combination thereof. The treatment may be an autologous or allogenic antigen specific T cell therapy.

In some embodiments, there is a method for monitoring the in vivo persistence of antigen specific T cells that have not been genetically modified and that have been administered to a subject, the method comprising (a) culturing peripheral blood mononuclear cells (PBMCs) obtained from the subject at a first time point (“baseline”) with a plurality of pepmix libraries, each library comprising a plurality of overlapping peptides that combine to cover a known tumor specific antigen (TSA); wherein culturing results in stimulating the expansion of one or more T cells contained within the PBMCs obtained from the subject; (b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries; (c) measuring whether the contacting in (b) results in an immune response elicited by the one or more T cells; wherein the measuring optionally comprises quantifying the magnitude of any immune response elicited by the one or more T cells in response to the contacting in (b); repeating steps (a) through (c) one or more additional times with PBMCs obtained from the subject at one or more subsequent time points; comparing the baseline immune response with one or more subsequent immune responses, wherein optionally the comparing comprises comparing the magnitude of the immune responses, wherein the detection of an immune response from one or more of the subsequent time points that is greater than or equal to the immune response generated by T cells obtained from the patient at the baseline time indicates that the antigen specific T cells still persist; and wherein the detection of an immune response from one or more of the subsequent time points that is less than the immune response generated by T cells obtained from the patient at the baseline time indicates that the antigen specific T cells no longer persist. In any methods of the disclosure, the tumor specific antigen is a neoantigen.

In certain embodiments, there are methods of treating subjects with cancer with an effective amount of one or more cancer therapies after the following: (a) culturing peripheral blood mononuclear cells (PBMCs) obtained from the subject in culture with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject; (b) contacting one or more of the T cells that have undergone expansion according to step (a) with (i) at least one of the pepmix libraries included in the said plurality of pepmix libraries, or (ii) at least one of the plurality of overlapping peptides contained in one of said pepmix libraries; (c) measuring that the contacting in (b) results in an immune response elicited by the one or more T cells. In specific embodiments, the cancer therapy comprises chemotherapy, immunotherapy, radiation, surgery, hormone therapy, or a combination thereof.

In some embodiments, there are methods of treating subjects with cancer with an effective amount of one or more cancer therapies after the following: (a) culturing memory T cells obtained from the subject with dendritic cells that have been contacted with a plurality of pepmix libraries, each library comprising a plurality of overlapping peptides that combine to cover a known tumor associated antigen (TAA); wherein culturing results in stimulating the expansion of one or more memory T cells obtained from the subject; (b) contacting one or more of the memory T cells that have undergone expansion according to step (a) with (i) at least one of the said pepmix libraries included in the plurality of pepmix libraries, or (ii) at least one of the plurality of overlapping peptides contained in one of said pepmix libraries; (c) measuring that the contacting in (b) results in an immune response elicited by the one or more T cells. In specific embodiments, the cancer therapy comprises chemotherapy, immunotherapy, radiation, surgery, hormone therapy, or a combination thereof.

### BRIEF DESCRIPTION OF THE DRAWINGS

FIG. 1 shows the T cell responses, assessed by IFNγ ELIspot, in AML patients that had received an allogeneic hematopoietic stem cell transplant and were tested for the presence of T cells directed against the tumor-expressed antigen cyclin-A1. Subsequently these patients were monitored for outcomes which revealed that those that remained disease-free (n=4) had significantly higher circulating tumor-specific T cell numbers than those who subsequently relapsed (n=4) (mean 171 vs 7.13 SFC/1×10⁵, p=0.03).

FIGS. 2A and 2B shows in vitro analysis of the expansion of multi-tumor-antigen targeted (multiTAA) T cells post-infusion in multiple myeloma patients in remission at the time of receiving T cell infusions. The frequency of functional tumor associated antigen (TAA)-specific T cells in peripheral blood was measured by an IFNγ ELISpot assay (FIG. 2A) and the persistence of infused T cells was monitored by TCR deep sequencing to track T cells derived from the multiTAA T cell lines infused (aka “unique clones”) in peripheral blood as well as the bone marrow (FIG. 2B).

FIGS. 3A and 3B shows in vitro analysis of multiTAA T cell expansion post-infusion in multiple myeloma patients with active disease as measured by assessing the frequency of functional TAA-specific T cells in peripheral blood using IFNγ ELISpot assay (FIG. 3A). MutliTAA-T cell-derived T cells were monitored by deep sequencing analysis and detected in peripheral blood as well as the bone marrow (FIG. 3B).

FIGS. 4A and 4B shows a cartoon demonstrating an exemplary diagnostic method according to the present disclosure. FIG. 4A shows a first memory T cell amplification method whereby PBMCs obtained from a patient having or suspected of having a cancer are cultured for 7-10 days in the presence of cytokines and one or more pepmix (each pepmix comprising a plurality of overlapping peptides that combine to span all or part of a TAA sequence) to stimulate proliferation of any circulating T cells that have specificity for one or more TAA peptide comprised in the pepmix(es). FIG. 4B shows a second memory T cell amplification method whereby memory cells obtained from a patient (e.g., isolated from PBMCs obtained from the patient) that has or is suspected of having a cancer are cultured for a minimum of 7-10 days in the presence of cytokines and antigen presenting cells APCs that have been contacted with one or more pepmix (each pepmix comprising a plurality of overlapping peptides that combine to span all or part of a TAA sequence) to stimulate proliferation of any circulating memory T cells that have specificity for one or more TAA peptide comprised in the pepmix(es). Following either one of the amplifications steps described in FIGS. 4A and 4B, a sample of the amplified memory T cells is spiked in a separate culture with one of the pepmixes, a peptide comprised in one of the pepmixes, and the resultant immune response is measured, e.g., via ELIspot analysis of IFNγ secretion or by TCR tracking of TAA-derived unique clones. The immune response mounted by these amplified memory T cells is used as a surrogate marker for prognosis, treatment decisions, and/or in the early diagnosis and treatment monitoring of cancer patients.

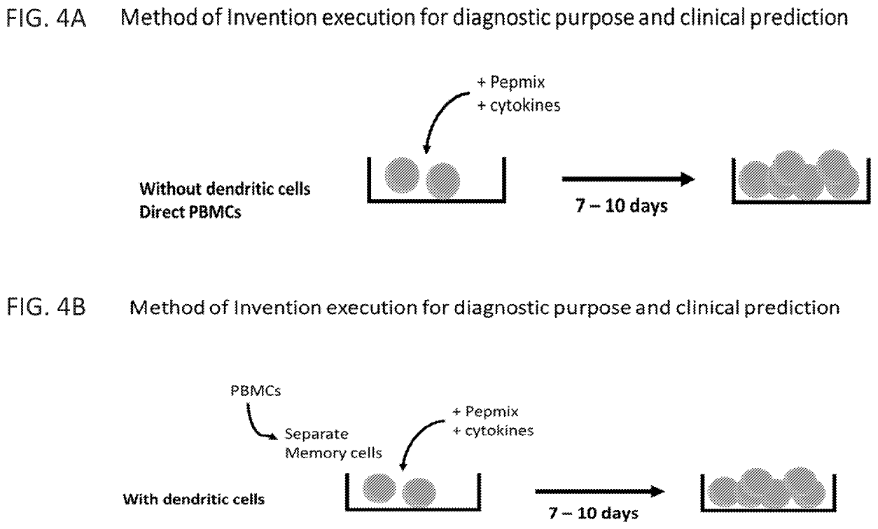

### DETAILED DESCRIPTION

### I. Definitions

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The term “about” when immediately preceding a numerical value means ±0% to 10% of the numerical value, ±0% to 10%, ±0% to 9%, ±0% to 8%, ±0% to 7%, ±0% to 6%, ±0% to 5%, ±0% to 4%, ±0% to 3%, ±0% to 2%, ±0% to 1%, ±0% to less than 1%, or any other value or range of values therein. For example, “about 40” means ±0% to 10% of 40 (i.e. from 36 to 44).

The use of the term “or” in the claims is used to mean “and/or” unless explicitly indicated to refer to alternatives only or the alternatives are mutually exclusive, although the disclosure supports a definition that refers to only alternatives and “and/or.”

The term “tumor associated antigen” as used herein refers to an antigenic substance produced/expressed on or in tumor cells and which triggers an immune response in the host.

Exemplary tumor associated antigens include at least the following: carcinoembryonic antigen (CEA) for bowel cancers; CA-125 for ovarian cancer; MUC-1 or epithelial tumor antigen (ETA) or CA15-3 for breast cancer; cyclin-A1 (CCNA1) for acute myeloid leukemia, tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myelom and in some lymphomas; CA19-9 for colorectal, bile duct, and pancreatic cancer; chromogranin A for lung and prostate cancer; TA90 for melanoma, soft tissue sarcomas, and breast, colon, and lung cancer. Examples of tumor antigens are known in the art, for example in Cheever et al., 2009, which is incorporated by reference herein in its entirety.

Specific examples of tumor antigens include at least CEA, MHC, CTLA-4, gp100, mesothelin, PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, cyclin A1 (CCNA1), Cyclin B1 (CCNB1), Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, folate receptor alpha (FR alpha), MAGE-A4, PRAME, and Fos-related antigen 1, for example. In some embodiments, the methods disclosed herein are extended to tumor specific antigens, e.g., mutated neoantigens such as KRAS, P53, BRAF, CDK4, MUM1, CTNNB1, CDC27, TRAPPC1, TPI, ASCC3, HHAT, FN1, OS-9, PTPRK, EF2, ACTN4, ME1, NF-YC for example by identifying hotspot sequences.

As used herein, the terms “patient” or “subject” are used interchangeably herein to refer to any mammal, including humans, domestic and farm animals, and zoo, sports, and pet animals, such as dogs, horses, cats, and agricultural use animals including cattle, sheep, pigs, and goats. One preferred mammal is a human, including adults, children, and the elderly. A subject may also be a pet animal, including dogs, cats and horses. Examples of agricultural animals include pigs, cattle and goats.

The terms “treat”, “treating”, “treatment” and the like, as used herein, unless otherwise indicated, refers to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the disease, condition, or disorder. In some instances, treatment is curative or ameliorating.

The terms “administering”, “administer”, “administration” and the like, as used herein, refer to any mode of transferring, delivering, introducing, or transporting a therapeutic agent to a subject in need of treatment with such an agent. Such modes include, but are not limited to, intraocular, oral, topical, intravenous, intraperitoneal, intramuscular, intradermal, intranasal, and subcutaneous administration.

As used herein, the terms “comprise,” “comprising,” “includes,” “including,” “has,” “having,” “contains,” “containing,” “characterized by,” or any other variation thereof, are intended to encompass a non-exclusive inclusion, subject to any limitation explicitly indicated otherwise, of the recited components. For example, a composition and/or method that “comprises” a list of elements (e.g., components or features or steps) is not necessarily limited to only those elements (or components or features or steps), but may include other elements (or components or features or steps) not expressly listed or inherent to the composition and/or method.

As used herein, the phrases “consists of” and “consisting of” exclude any element, step, or component not specified. For example, “consist of” or “consisting of” used in a claim would limit the claim to the components, materials or steps specifically recited in the claim except for impurities ordinarily associated with therewith (i.e. impurities within a given component). When the phrase “consist of” or “consisting of” appears in a clause of the body of a claim, rather than immediately following the preamble, the phrase “consist of” or “consisting of” limits only the elements (or components or steps) set forth in that clause; other elements (or components) are not excluded from the claim as a whole.

Other objects, feature and advantages of the present invention will become apparent from the following detailed description. It should be understood, however, that the detailed description and specific examples, while indicating specific embodiments of the invention, are given by way of illustration only, since various changes and modifications within the spirit and scope of the invention will become apparent to those skilled in the art from this detailed description.

### II. Overview

The present disclosure provides, inter alia, compositions and methods for harnessing the immune system (e.g., T cells) as a detection tool to diagnose and predict cancer patient treatment outcomes. The invention is based in part on the surprising discovery that although TAAs are poor stimulators of the immune system (as they generally represent some variation of a self-antigen), we can nevertheless detect circulating T cells with specificity for tumor associated antigens with a simple in vitro amplification step. And, the presence of these circulating tumor-specific T cells is indicative of the presence of a tumor in the patient.

Thus, in some embodiments, the present disclosure provides a method of diagnosing the presence of a tumor in a subject comprising amplifying T cells with specificity for one or more known tumor associate antigen (TAA) from peripheral blood mononuclear cells (PBMCs) obtained from the subject, and then testing the amplified T cells for TAA specificity. In various embodiments, the amplification is accomplished in one of two different ways. Firstly, in one embodiment, PBMCs obtained from the patient are stimulated in culture with suitable cytokines by directly culturing the PBMCs with one or more pepmixes, each of which comprises a plurality of peptides with partially overlapping sequences that together combine to span a selected TAA sequence (or a portion thereof), for a minimum of 5 days, this sample are then divided in individual cell culture wells containing peptide sequences that represent the individual antigens present in the original peptide mixture and evaluate the biological response by measuring the production of IFNg after 4 hours and no later than 72 hours. Secondly, in one embodiment, T cells present in PBMCs obtained from the patient are stimulated in culture with antigen presenting cells primed with one or more pepmixes, each of which comprises a plurality of synthetic peptides of partially overlapping sequences that together combine to span a selected TAA sequence (or a portion thereof). T cells within PBMCs obtained from the patient that have specificity for one or more of the peptides in the pepmixes are specifically amplified (via stimulation based proliferation), and their presence may then be detected by any suitable method known in the art or disclosed herein.

A single pepmix (covering a single antigen or portion thereof) may be used in either one of the two amplification methods described above. However, we have demonstrated in our prior work that multiple pepmixes may also be combined without preventing the generation of multi-TAA specific T cells (WO 2011/02853 and WO 2013/119947, each of which is incorporated herein by reference in its entirety); thus, in some embodiments a plurality of pepmixes are pooled and used simultaneously in the amplification step to stimulate proliferation of a plurality of memory T cell clones in a single culture, each pepmix comprising a library of overlapping synthetic peptides spanning a different tumor associated antigen sequence (or an epitope portion thereof).

The presence of T cells (e.g., memory T cells) amplified via the first or second amplification methods described above may in some embodiments be determined by re-contacting (or “spiking”) the amplified T cells in culture with the pepmixes that were used during amplification step. Additionally or alternatively, the presence of the T cells (e.g., memory T cells) amplified via the first or second amplification methods described above may in some embodiments be determined by re-contacting (or “spiking”) the amplified T cells in culture with one or more isolated peptides that were comprised in the pepmixes, to induce an immune response. The immune response may be detected, e.g., by monitoring resultant cytokine secretion (e.g., secretion of interferon-γ) via a suitable assay such as an ELIspot assay or, e.g., by analyzing the cells for markers of T cell activation via a suitable assay such as flow cytometry. In some embodiments, spiking the amplified T cells in culture with isolated peptides from the pepmixes (i.e. spiking with a single peptide sequence at a suitable concentration), rather than spiking with a plurality of peptides or a plurality of pepmixes, enables determination of the exact TAA sequence that is being recognized by the T cells. In some embodiments, this is to be used as a correlate for understanding the expression profile of the immunogenic epitopes expressed on the tumor. Similarly, spiking with a pepmix comprising a plurality of peptides with partially overlapping sequences that together combine to span a single selected TAA sequence (or a portion thereof), enables determination of the exact TAA that is being recognized by the T cells. In some embodiments, aliquots of the amplified T cells (e.g., memory T cells) are seeded in separate cultures and then these cultures are spiked with a test pepmix that was used in the amplification step (or a peptide from the pepmix), and any resultant immune responses are measured as described above. These separate cultures may be in, e.g., a format suitable for high throughput analysis, e.g., a 96 well or 384 well culture plate. In some embodiments, a plurality of pepmixes each covering a different TAA are used to amplify the T cells using a method described herein, aliquots of the amplified T cells are seeded in separate cultures, each separate culture is spiked with one of the pepmixes utilized in the amplification step, a single peptide included in one of the pepmixes, or a suitable control, and any resultant immune responses are measured as described above (optionally in a high throughput setting). In this way, numerous TAA pepmixes may be used together to stimulate the amplification of circulating memory T cells resulting in a milieu of amplified T cells, and then the specificity of these amplified T cells may be deconvoluted during the measuring step, as any resultant immune responses (e.g., production of IFNγ) is indicative of the presence of T cell specificity for a particular TAA (if an entire pepmix is used in the spiking step) or that specific peptide epitope (if a single peptide from the pepmix is used in the spiking step) on the tumor. Thus, in some embodiments, immune responses detected by the above methods are used as a correlate for understanding the expression profile of the TAAs expressed on the tumor.

Additionally, in some embodiments, the above methods provide information about the tumor and/or about the patient's immune system that aid in predicting whether a patient will relapse following treatment with an anti-tumor therapy. For example, as shown in Example 1, AML patients in remission have endogenous circulating memory T cells with specificity for CCNA1, a known AML TAA (Ochsenreither, S, et al., Blood. 2012 Jun. 7; 119(23): 5492-5501, incorporated herein by reference in its entirety), and these T cells are detectable using the methods disclosed herein. Surprisingly, the levels of these circulating memory T cells fall into two categories in these patients: high and low levels; and the patients with low levels were statistically more likely to relapse than the patients with high levels. Thus, in one embodiment, the present disclosure provides a method of predicting whether a patient is likely to relapse, the method comprising amplifying the T cells via one of the amplification methods discussed herein; detecting the presence of circulating T cells with specificity for a TAA (e.g., CCNA1) by spiking the amplified T cells in culture with the TAA or a pepmix comprising a plurality of peptides with partially overlapping sequences that together combine to span a selected TAA sequence (or a portion thereof)(e.g., CCNA1) or with one or more peptide comprised in such a pepmix; quantifying the magnitude of the response; and comparing the magnitude of the response with (i) a response generated in a similar method using one or more reference samples from subjects with known treatment outcomes (i.e. known to have relapsed, or not) or (ii) historical response values from one or more reference samples, such as a plurality of such reference samples, from subjects with known treatment outcomes (i.e. known to have relapsed, or not); wherein if the patient has high levels of circulating T cells with specificity against one or more TAA the patient is determined to have a good prognosis (i.e. is likely to not relapse) and wherein if the patient has low levels of circulating memory T cells with specificity against one or more TAA or against all TAAs tested, the patient is determined to have a poor prognosis (i.e. is likely to relapse). Patients determined to have a good prognosis are in some embodiments selected for further monitoring of the tumor via a tumor monitoring method described herein or known in the art. Patients determined to have a poor prognosis are in some embodiments selected for further treatment. Treatment may include without limitation chemotherapy, immunotherapy, radiation therapy, resection surgery, transplant (solid tissue or stem cell) or a combination thereof. Treatment may also include further monitoring of the tumor via a tumor monitoring method described herein or known in the art.

### Tumor Monitoring Methods

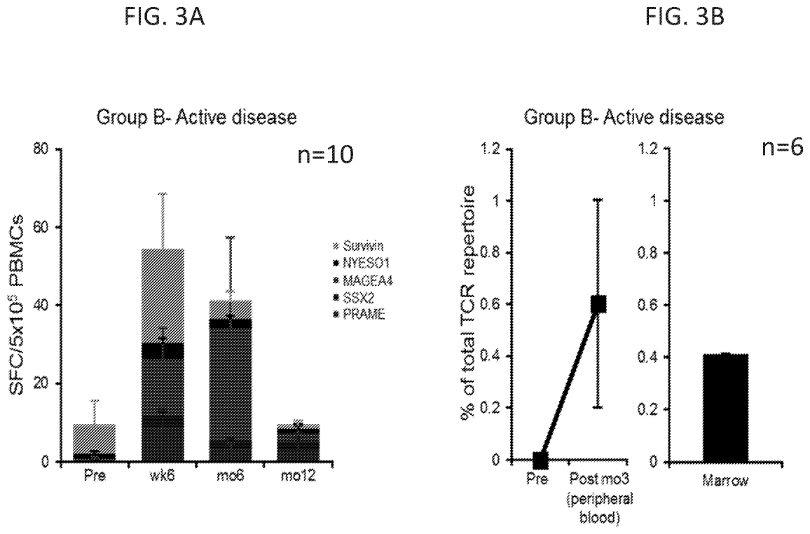

Moreover, in some embodiments, the above methods are performed on serial samples obtained from the subject over the course of a period (e.g., over a period of months or years; before, during, and/or after receipt of a treatment, etc.) and temporal changes in the populations of circulating T cells, e.g., memory T cells (i.e. changes in the quantities or identities of TAA-specific T cells) are observed based on changes in the magnitude of immune responses generated by the amplified T cells against a given antigen or panel of antigens. Such changes are used in some embodiments to monitor the TAA expression profile of a tumor over time, e.g., in the context of a treatment paradigm or in the context of a wait and see approach whereby changes in the magnitude of circulating T cells with specificity against TAAs expressed by the tumor may signal growth of the tumor or antigen escape. As TAA-specific memory T cells arise in response even to microscopic tumors, this information is useful for the early diagnosis and treatment monitoring of cancer patients. Moreover, these methods may be used to monitor the persistence of autologous or allogeneic antigen specific T cells that have been previously administered to a patient in connection with an immunotherapy. Further, these methods may be used to specifically monitor functional autologous or allogeneic antigen specific T cells. In some embodiments, such T cells are not genetically modified. For example, as shown in Example 2, exogenously delivered T cell products with specificity for multiple TAAs (“multiTAA-specific CTLs”) were administered to individuals with active or inactive multiple myeloma (MM) and the above detection methods were used to monitor circulating levels of these multiTAA-specific CTLs prior to administration and at various time points post administration. As used herein the term “CTL” refers to a cytotoxic T cell. Over time, the multiTAA-specific CTLs amplified significantly in patients with active disease (FIG. 3), but only slightly in patients with inactive disease. These data demonstrate that in some embodiments, temporal increases in multiTAA-specific CTLs for a given TAA signal the presence of active disease and subsequent temporal decreases in the multiTAA-specific CTLs for a given TAA in some embodiments signal that the treatment has effectively controlled the tumor or that the tumor has undergone antigen escape. In some embodiments, by analyzing a plurality of TAAs via the methods disclosed herein, one can deconvolute these potentially disparate outcomes. For example, temporal disappearance of one or a few, but not all multiTAA-specific CTLs that bind TAAs on a given tumor may indicate antigen escape. Alternatively, disappearance of all multiTAA-specific CTLs that bind TAAs on a given tumor may indicate that the tumor has been effectively controlled by treatment and the patient is in remission. These measurements may also be coupled with appropriate positive controls to rule out the possibility that the disappearance of all multiTAA-specific CTLs that bind TAAs on a given tumor indicates that the patient is merely experiencing a generally compromised immune system.

Moreover, in some embodiments, a large panel of pepmixes covering a large plurality of TAAs, (e.g., 10-100 TAAs, or 30-50 TAAs or 20 to 40 TAAs) are utilized in the amplification and subsequent spiking steps described above across a plurality of time-points temporally (e.g., samples obtained before and after treatment and at subsequent weeks or months thereafter) and changes in the profile of circulating TAA-specific CTLs that are detectable in the samples from the subject can inform on treatment paradigms. For example, if the numbers of TAA-specific CTLs that target a particular TAA are detected in a subject either in an initial time-point or if such CTLs increase in number over time in the patient (as measured via the above methods) this may inform that a therapy such as, e.g., an immunotherapy (e.g., a chimeric antigen receptor T cell therapy or allogeneic or autologous T cell therapy) that targets a particular antigen may be likely to result in efficacious treatment. Similarly, lack of any circulating TAA-specific CTLs that target a particular tumor antigen may indicate in some embodiments that treatment with a therapy such as, e.g., an immunotherapy (e.g., a chimeric antigen receptor T cell therapy or allogeneic or autologous T cell therapy) that targets the particular antigen may be contraindicated.

Similarly, in alternative embodiments, each of the methods described herein may be performed as described herein, except that instead of relating to a TAA by amplifying TAA-specific T cells and detecting TAA-specific T cells, such methods relate to amplifying T cells with specificity for one or more known tumor specific antigen (TSA), e.g., a neoantigen, from peripheral blood mononuclear cells (PBMCs) obtained from the subject, and then testing the amplified T cells for TSA (e.g., neoantigen) specificity. In various embodiments, the TSA (e.g., neoantigen) T cell amplification is accomplished using one of the two different amplification methods described herein.

In alternative embodiments, the pepmixes may be substituted in the two amplification steps described above with a whole TAA antigen, e.g., by administering a suitable concentration of a purified antigen (such as a whole TAA protein or an antigenic portion thereof) or by transfecting dendritic cells with an expression construct for the TAA protein or an antigenic portion thereof. Similarly, the spiking steps described above may utilize whole TAA antigen, e.g., by administering a suitable concentration of a purified antigen (such as a whole TAA protein or an antigenic portion thereof) or may comprise incubating the amplified T cells with another cell presenting the TAAs or portions thereof, e.g., dendritic cells transfected with an expression construct for the TAA protein or an antigenic portion thereof. Similarly, in some embodiments, the pepmixes utilized in the subsequent spiking steps that follow the amplification steps may be substituted with a whole TAA antigen sequence encoded by DNA or RNA and incorporated into the APCs by electroporation, nucleofection, lipofection or passive uptake e.g.

### III. Tumor Antigens

In embodiments wherein multiTAA-specific CTL are employed for the treatment and/or prevention of cancer, a variety of TAA may be targeted. Tumor antigens are substances produced in tumor cells that trigger an immune response in a host.

Exemplary tumor antigens include at least the following: carcinoembryonic antigen (CEA) for bowel cancers; CA-125 for ovarian cancer; MUC-1 or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myelom and in some lymphomas; CA19-9 for colorectal, bile duct, and pancreatic cancer; chromogranin A for lung and prostate cancer; TA90 for melanoma, soft tissue sarcomas, and breast, colon, and lung cancer. Examples of tumor antigens are known in the art, for example in Cheever et al., 2009, which is incorporated by reference herein in its entirety.

Specific examples of tumor antigens include at least CEA, MHC, CTLA-4, gp100, mesothelin, PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, folate receptor alpha (FR alpha), MAGE-A4, PRAME, and Fos-related antigen 1, for example.

### IV. Generation of Pepmix Libraries

In some embodiments of the invention, a library of peptides is provided to PBMCs or dendritic cells ultimately to generate CTLs. The library in particular cases comprises a mixture of peptides (“pepmixes”) that span part or all of the same antigen or a portion thereof. Pepmixes utilized in the invention may be from commercially available peptide libraries. In some embodiments, pepmixes utilized in the invention comprise a plurality of peptides that are 15 amino acids long and overlap one another by 11 amino acids, such that each peptide walks along all or part of a particular antigen. In some cases, pepmixes may be generated synthetically. Examples include those from JPT Technologies (Springfield, Va.) or Miltenyi Biotec (Auburn, Calif.). In particular embodiments, the peptides in the pepmixes are at least 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and in specific embodiments there is overlap of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length, for example. The mixture of different peptides may include any ratio of the different peptides, although in some embodiments each particular peptide is present at substantially the same numbers in the mixture as another particular peptide.

### V. Culture Conditions

In some embodiments of the disclosure, tumor-targeted T cells can be generated by directly exposing PBMCs to antigenic pepmixes in the presence of one or more activating cytokines such as IL7, IL12, IL6, IL15, and IL2 or by co-culturing pepmix-loaded dendritic cells with PBMCs in the presence of one or more activating cytokines such as IL7, IL12, IL6, IL15, and IL2. Cells are cultured for 7-14 days under these conditions in plasticware such as 24-well plates or G-Rex devices. T cells may undergo subsequent rounds of stimulation by co-culture with antigen-loaded APCs (either irradiated or non-irradiated) with antigen-activated T cells in the presence of pro-proliferative cytokines such as IL15, IL2, or combinations thereof. One or more activating cytokines such as IL7, IL12, IL6, IL15, and IL2 may be utilized.

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Detection of Circulating (Endogenous) CCNA1-Reactive Cells in Individuals with AML Who had Received an Allo-HSCT and were in Remission

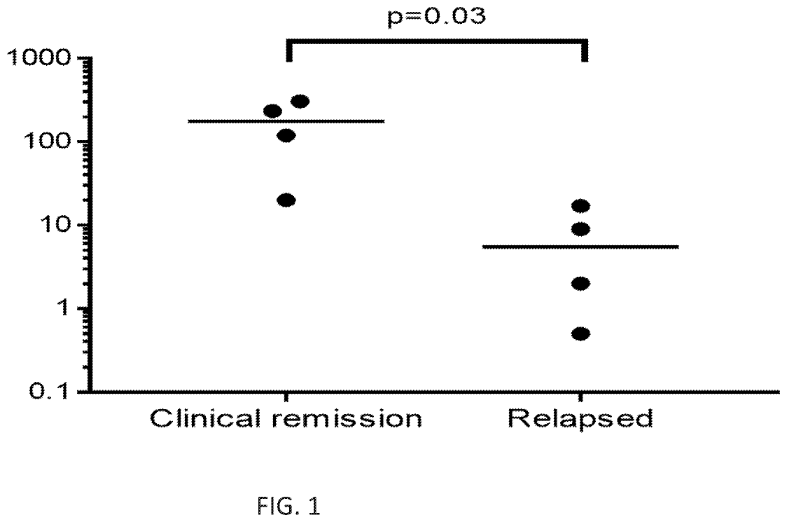

To determine whether individuals with AML who had received an allo-HSCT and were in remission might have circulating (endogenous) CCNA1-reactive cells capable of mediating protective anti-tumor effects, we stimulated patient-derived PBMCs (n=8) with CCNA1 using samples that were collected at 2-3 months post-transplant while all were in remission. We then followed these patients for clinical outcomes and found a direct correlation between the presence and magnitude of CCNA1-directed T cells measured early post-transplant and risk of disease relapse by 1 year post-HSCT. FIG. 1 shows the T cell responses, assessed by IFNγ ELIspot, in relapse-free patients (n=4) compared with those who subsequently relapsed (n=4) (mean 171 vs 7.13 SFC/1×10⁵, p=0.03). These findings demonstrate the protective benefit conferred by endogenous CCNA1-specific T cells post-HSCT, as patients that eventually relapsed were found to have lower circulating CCNA1-specific T cells while they were in remission than patients that remained in remission 1 year post-HSCT. Thus, these data illustrate that the magnitude of circulating TAA-specific T cells during remission can act as a prognosticator for relapse.

### Example 2. Detection of Circulating MultiTAA T Cells (Exogenous) in Individuals with Active or Inactive Multiple Myeloma (MM)

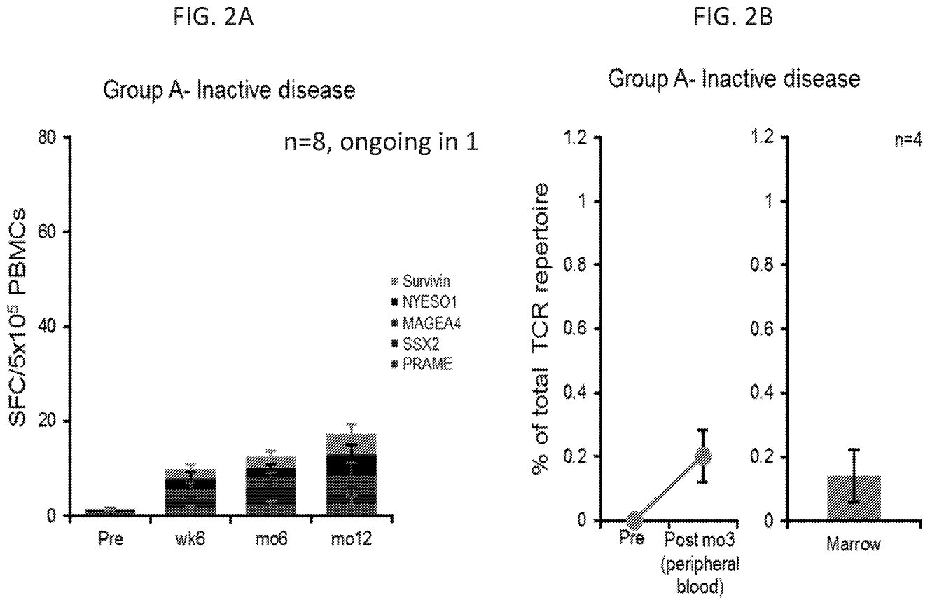

To determine whether circulating multiTAA T cells were detectable and persisted in multiple myeloma patients infused with ex vivo expanded T cells targeting multiple tumor associated antigens (multiTAA T cells) as adjuvant therapy, we stimulated patient-derived PBMCs (n=8) obtained from these patients before infusion and after infusion at week 6, month 6, and month 12 with pepmixes covering the five tumor associated antigens (TAAs) to which the multiTAA T cells were raised, and amplified for 7 to 10 days, after this amplification we measured resultant immune reactions by ELIspot detection of antigen-specific IFNγ secretion and by antigen-specific responses TCR tracking of multiTAA-derived “unique clones.” Two separate patient groups were studied: those with active disease (Group A) and those with inactive disease (Group B). Antigen-specific responses were detectable in both groups. However, only modest peripheral T cell expansion was observed in patients with inactive disease (increase from 1.31 to 9.6 SFCs; 0 to 0.32±0.18% repertoire) with low level detection in the bone marrow (0.49±0.03%) (FIG. 2A, 2B). In contrast, overall, in the 10 multiple myeloma patients who were administered multiTAA T cells to treat active disease, all had a concomitant increase in circulating tumor-reactive T cells (2.8-28.7 fold mean increase based on ELIspot detection of antigen-specific responses) (FIG. 3A). The in vivo expansion was evident not only by IFNγ assessment but also by TCR tracking of multiTAA-derived “unique clones” (increase from 0% at baseline to a mean of 0.6±0.4% of the entire T cell repertoire), with confirmed infiltration into the bone marrow (0.99±0.78%) when assessed 8 to 12 weeks post-infusion (FIG. 3B). These data confirm that exogenous multiTAA T cells persist in vivo and amplify in patients with active disease. Thus, these data support the use of the methods described herein for monitoring the presence of circulating T cells with specificity for known TAAs and using the presence or absence of such T cells as a readout of whether a patient's disease is active or inactive. Moreover, changes over time in the abundance of these circulating T cells during subsequent treatment may inform on whether the treatment is working.

1. A method of diagnosing the presence of a tumor in a subject comprising:
(a) culturing peripheral blood mononuclear cells (PBMCs) obtained from the subject in culture with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject;
(b) contacting one or more of the T cells that have undergone expansion according to step (a) with (i) at least one of the pepmix libraries included in the said plurality of pepmix libraries, or (ii) at least one of the plurality of overlapping peptides contained in one of said pepmix libraries;
(c) measuring whether the contacting in (b) results in an immune response elicited by the one or more T cells; and
(d) diagnosing the subject as having the tumor if the contacting in (b) does result in an immune response as measured in (c).

2. A method of diagnosing the presence of a tumor in a subject comprising:
a. culturing memory T cells obtained from the subject with dendritic cells that have been contacted with a plurality of pepmix libraries, each library comprising a plurality of overlapping peptides that combine to cover a known tumor associated antigen (TAA); wherein culturing results in stimulating the expansion of one or more memory T cells obtained from the subject;
b. contacting one or more of the memory T cells that have undergone expansion according to step (a) with (i) at least one of the said pepmix libraries included in the plurality of pepmix libraries, or (ii) at least one of the plurality of overlapping peptides contained in one of said pepmix libraries;
c. measuring whether the contacting in (b) results in an immune response elicited by the one or more T cells; and
d. diagnosing the subject as having the tumor if the contacting in (b) does result in an immune response as measured in (c).

3. The method of claim 2, wherein the T cells are isolated from PBMCs obtained from the subject.

2. The method of any one of the preceding claims, wherein the culturing step (a) covers a period of 7-10 days.

3. The method of any one of the preceding claims, wherein the cells are in the presence of at least 2, 3, 4, 5, 6, 7, 8, 9, 10, 11, 12, 13, 14, 15, 16, 17, 18, 19, 20, or more different pepmixes, each pepmix comprising a series of overlapping peptides that span part of or the entire sequence of an antigen.

4. The method of any one of the preceding claims, wherein at least 2, 3, 4, 5, 6, 7, 8, 9, 10, 11, 12, 13, 14, 15, 16, 17, 18, 19, 20 or more different antigens are covered by the plurality of pepmixes.

5. The method of any one of the preceding claims, wherein the pepmix comprises 15 mer peptides.

6. The method of any one of the preceding claims, wherein the pepmix comprises 7 mer, 8 mer, 9 mer, 10 mer, 11 mer, 12 mer, 13 mer, 14 mer, or 15 mer or greater peptides.

7. The method of any one of the preceding claims, wherein the peptides in the pepmix that span the antigen overlap in sequence by 7, 8, 9, 10, 11, 12, 13, or 14 or more amino acids.

8. A method of predicting whether a cancer patient is likely to respond to multi TAA T-cell therapy, comprising:
(a) performing steps (a) through (c) set forth in claim 1; quantifying the magnitude of the immune response from step (c); and comparing the magnitude of the patient's immune response with a reference immune response selected from
(i) a response generated in a similar method using one or more reference samples from subjects with known treatment outcomes (i.e. known to have relapsed, or not) or
(ii) historical response values from one or more reference samples from subjects with known treatment outcomes (i.e. known to have relapsed, or not);
wherein (A) if the immune response from step (c) is comparable to the reference immune response of a patient known to have relapsed, the patient is determined to have a high likelihood of relapsing; and (B) if the immune response from step (c) is comparable to the reference immune response of a patient known to not have relapsed, the patient is determined to have a low likelihood of relapsing.

9. The method of claim 8, wherein if the patient is determined to have a high likelihood of relapsing, the patient is treated with chemotherapy, immunotherapy, radiation therapy, resection surgery, transplant (solid tissue or stem cell) or a combination thereof.

10. The method of claim 8, wherein if the patient is determined to have a low likelihood of relapsing, steps (a) through (c) set forth in claim 1 are repeated one or more additional times over time to monitor whether any temporal changes in the immune responses elicited by the one or more T cells is detectable.

11. The method of claim 10, wherein if a temporal change in the immune responses elicited by the one or more T cells is detectable, the magnitude of the change is quantified and compared one or more time to the magnitude of the immune response of a reference immune response as set forth in claim 8.

12. The method of claim 11, wherein if the patient is determined to have a high likelihood of relapsing after one or more time of comparing the magnitude of patient's immune response with a reference immune response, the patient is treated with chemotherapy, immunotherapy, radiation therapy, resection surgery, transplant (solid tissue or stem cell) or a combination thereof.

13. A method for detecting antigen loss comprising:
(a) culturing peripheral blood mononuclear cells (PBMCs) obtained from the subject at a first time point (“baseline”) with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject;
(b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries;
(c) measuring and quantifying the magnitude of any immune response elicited by the one or more T cells in response to the contacting in (b);
(d) repeating steps (a) through (c) one or more additional times with PBMCs obtained from the subject at one or more subsequent time points;
(e) comparing the magnitude of the immune responses from the first and one or more of each subsequent quantifying step; and
(f) identifying any immune responses that change in magnitude over time; wherein a change in an immune response elicited by a particular pepmix covering a known TAA, or by a peptide comprised in such a pepmix, indicates a change in the expression of that TAA by a tumor in the patient.

14. The method of claim 13, wherein the first time point is before the patient receives a treatment for the cancer and each subsequent time point is after the patient receives the treatment.

15. The method of claim 13 or 14, wherein the first time point and a first subsequent time point are separated by a period ranging from one week to one year, one week to six months or one week to one month.

16. The method of any one of claims 13-15 comprising at least 2 subsequent time points.

17. The method of any one of claims 13-16 comprising 2, 3, 4, 5, 6, 7, 8, 9, 10, 11, 12, 13, 14, 15, 16, 17, 18, 19, or 20 subsequent time points.

18. The method of any one of claims 13-17, wherein the treatment is selected from a chemotherapy, immunotherapy, radiation therapy, resection surgery, solid tissue transplant, a stem cell transplant, or a combination thereof.

19. The method of any one of claims 13-17, wherein the treatment is an autologous or allogenic antigen specific T cell therapy.

20. A method of predicting whether a patient is likely to relapse after a remission, comprising:
(a) performing steps (a) through (c) set forth in claim 1; quantifying the magnitude of the immune response from step (c); and comparing the magnitude of the patient's immune response with a reference immune response selected from
(iii) a response generated in a similar method using one or more reference samples from subjects with known treatment outcomes (i.e. known to have relapsed, or not) or
(iv) historical response values from one or more reference samples from subjects with known treatment outcomes (i.e. known to have relapsed, or not);
wherein (A) if the immune response from step (c) is comparable to the reference immune response of a patient known to have relapsed, the patient is determined to have a high likelihood of relapsing; and (B) if the immune response from step (c) is comparable to the reference immune response of a patient known to not have relapsed, the patient is determined to have a low likelihood of relapsing.

21. The method of claim 20, wherein if the patient is determined to have a high likelihood of relapsing, the patient is treated with chemotherapy, immunotherapy, radiation therapy, resection surgery, transplant (solid tissue or stem cell) or a combination thereof.

22. The method of claim 20, wherein if the patient is determined to have a low likelihood of relapsing, steps (a) through (c) set forth in claim 1 are repeated one or more additional times over time to monitor whether any temporal changes in the immune responses elicited by the one or more T cells is detectable.

23. The method of claim 22, wherein if a temporal change in the immune responses elicited by the one or more T cells is detectable, the magnitude of the change is quantified and compared one or more time to the magnitude of the immune response of a reference immune response as set forth in claim 8.

24. The method of claim 23, wherein if the patient is determined to have a high likelihood of relapsing after one or more time of comparing the magnitude of patient's immune response with a reference immune response, the patient is treated with chemotherapy, immunotherapy, radiation therapy, resection surgery, transplant (solid tissue or stem cell) or a combination thereof.

25. A method for detecting changes in the TAA expression profile of a tumor over time comprising:
(a) culturing peripheral blood mononuclear cells (PBMCs) obtained from the subject at a first time point (“baseline”) with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject;
(b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries;
(c) measuring and quantifying the magnitude of any immune response elicited by the one or more T cells in response to the contacting in (b);
(d) repeating steps (a) through (c) one or more additional times with PBMCs obtained from the subject at one or more subsequent time points;
(e) comparing the magnitude of the immune responses from the first and one or more of each subsequent quantifying step; and
(f) identifying any immune responses that change in magnitude over time; wherein a change in an immune response elicited by a particular pepmix covering a known TAA, or by a peptide comprised in such a pepmix, indicates a change in the expression of that TAA by a tumor in the patient.

26. The method of claim 25, wherein the first time point is before the patient receives a treatment for the cancer and each subsequent time point is after the patient receives the treatment.

27. The method of claim 25 or 26, wherein the first time point and a first subsequent time point are separated by a period ranging from one week to one year, one week to six months or one week to one month.

28. The method of any one of claims 25-27 comprising at least 2 subsequent time points.

29. The method of any one of claims 25-28 comprising 2, 3, 4, 5, 6, 7, 8, 9, 10, 11, 12, 13, 14, 15, 16, 17, 18, 19, or 20 subsequent time points.

30. The method of any one of claims 25-29, wherein the treatment is selected from a chemotherapy, immunotherapy, radiation therapy, resection surgery, solid tissue transplant, a stem cell transplant, or a combination thereof.

31. The method of any one of claims 25-29, wherein the treatment is an autologous or allogenic antigen specific T cell therapy.

32. A method for monitoring the in vivo persistence of antigen specific T cells that have not been genetically modified and that have been administered to a subject, the method comprising
(a) culturing peripheral blood mononuclear cells (PBMCs) obtained from the subject at a first time point (“baseline”) with a plurality of pepmix libraries, each library comprising a plurality of overlapping peptides that combine to cover a known tumor associated antigen (TAA); wherein culturing results in stimulating the expansion of one or more T cells contained within the PBMCs obtained from the subject;
(b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries;
(c) measuring whether the contacting in (b) results in an immune response elicited by the one or more T cells; wherein the measuring optionally comprises quantifying the magnitude of any immune response elicited by the one or more T cells in response to the contacting in (b);
(d) repeating steps (a) through (c) one or more additional times with PBMCs obtained from the subject at one or more subsequent time points;
(e) comparing the baseline immune response with one or more subsequent immune responses, wherein optionally the comparing comprises comparing the magnitude of the immune responses,
(f) wherein the detection of an immune response from one or more of the subsequent time points that is greater than or equal to the immune response generated by T cells obtained from the patient at the baseline time indicates that the antigen specific T cells still persist;
(g) and wherein the detection of an immune response from one or more of the subsequent time points that is less than the immune response generated by T cells obtained from the patient at the baseline time indicates that the antigen specific T cells no longer persist.

33. A method of diagnosing the presence of a tumor in a subject comprising:
(a) culturing peripheral blood mononuclear cells (PBMCs) obtained from the subject in culture with a plurality of pepmix libraries, each library comprising a plurality of overlapping peptides that combine to cover a known tumor specific antigen (TSA); wherein culturing results in stimulating the expansion of one or more T cells contained within the PBMCs obtained from the subject;
(b) contacting one or more of the T cells that have undergone expansion according to step (a) with (i) at least one of the pepmix libraries included in the said plurality of pepmix libraries, or (ii) at least one of the plurality of overlapping peptides contained in one of said pepmix libraries;
(c) measuring whether the contacting in (b) results in an immune response elicited by the one or more T cells; and
(d) diagnosing the subject as having the tumor if the contacting in (b) does result in an immune response as measured in (c).

34. A method of diagnosing the presence of a tumor in a subject comprising:
(a) culturing memory T cells obtained from the subject with dendritic cells that have been contacted with a plurality of pepmix libraries, each library comprising a plurality of overlapping peptides that combine to cover a known tumor specific antigen (TSA); wherein culturing results in stimulating the expansion of one or more memory T cells obtained from the subject;
(b) contacting one or more of the memory T cells that have undergone expansion according to step (a) with (i) at least one of the said pepmix libraries included in the plurality of pepmix libraries, or (ii) at least one of the plurality of overlapping peptides contained in one of said pepmix libraries;
(c) measuring whether the contacting in (b) results in an immune response elicited by the one or more T cells; and
(d) diagnosing the subject as having the tumor if the contacting in (b) does result in an immune response as measured in (c).

35. The method of claim 34, wherein the T cells are isolated from PBMCs obtained from the subject.

36. The method of any one of the preceding claims, wherein the culturing step (a) covers a period of 7-10 days.

37. The method of any one of the preceding claims, wherein the cells are in the presence of at least 2, 3, 4, 5, 6, 7, 8, 9, 10, 11, 12, 13, 14, 15, 16, 17, 18, 19, 20, or more different pepmixes, each pepmix comprising a series of overlapping peptides that span part of or the entire sequence of an antigen.

38. The method of any one of the preceding claims, wherein at least 2, 3, 4, 5, 6, 7, 8, 9, 10, 11, 12, 13, 14, 15, 16, 17, 18, 19, 20 or more different antigens are covered by the plurality of pepmixes.

39. The method of any one of the preceding claims, wherein the pepmix comprises 15 mer peptides.

40. The method of any one of the preceding claims, wherein the peptides in the pepmix that span the antigen overlap in sequence by 11 amino acids.

41. A method of predicting whether a cancer patient is likely to respond to multi TSA T-cell therapy, comprising:
(a) performing steps (a) through (c) set forth in claim 1; quantifying the magnitude of the immune response from step (c); and comparing the magnitude of the patient's immune response with a reference immune response selected from
(v) a response generated in a similar method using one or more reference samples from subjects with known treatment outcomes (i.e. known to have relapsed, or not) or
(vi) historical response values from one or more reference samples from subjects with known treatment outcomes (i.e. known to have relapsed, or not);
wherein (A) if the immune response from step (c) is comparable to the reference immune response of a patient known to have relapsed, the patient is determined to have a high likelihood of relapsing; and (B) if the immune response from step (c) is comparable to the reference immune response of a patient known to not have relapsed, the patient is determined to have a low likelihood of relapsing.

42. The method of claim 39, wherein if the patient is determined to have a high likelihood of relapsing, the patient is treated with chemotherapy, immunotherapy, radiation therapy, resection surgery, transplant (solid tissue or stem cell) or a combination thereof.

43. The method of claim 40, wherein if the patient is determined to have a low likelihood of relapsing, steps (a) through (c) set forth in claim 1 are repeated one or more additional times over time to monitor whether any temporal changes in the immune responses elicited by the one or more T cells is detectable.

44. The method of claim 41, wherein if a temporal change in the immune responses elicited by the one or more T cells is detectable, the magnitude of the change is quantified and compared one or more time to the magnitude of the immune response of a reference immune response as set forth in claim 8.

45. The method of claim 41, wherein if the patient is determined to have a high likelihood of relapsing after one or more time of comparing the magnitude of patient's immune response with a reference immune response, the patient is treated with chemotherapy, immunotherapy, radiation therapy, resection surgery, transplant (solid tissue or stem cell) or a combination thereof.

46. A method for detecting antigen loss comprising:
(a) culturing peripheral blood mononuclear cells (PBMCs) obtained from the subject at a first time point (“baseline”) with a plurality of pepmix libraries, each library comprising a plurality of overlapping peptides that combine to cover a known tumor specific antigen (TSA); wherein culturing results in stimulating the expansion of one or more T cells contained within the PBMCs obtained from the subject;
(b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries;
(c) measuring and quantifying the magnitude of any immune response elicited by the one or more T cells in response to the contacting in (b);
(d) repeating steps (a) through (c) one or more additional times with PBMCs obtained from the subject at one or more subsequent time points;
(e) comparing the magnitude of the immune responses from the first and one or more of each subsequent quantifying step; and
(f) identifying any immune responses that change in magnitude over time; wherein a change in an immune response elicited by a particular pepmix covering a known TSA, or by a peptide comprised in such a pepmix, indicates a change in the expression of that TSA by a tumor in the patient.

47. The method of claim 44, wherein the first time point is before the patient receives a treatment for the cancer and each subsequent time point is after the patient receives the treatment.

48. The method of claim 44 or 45, wherein the first time point and a first subsequent time point are separated by a period ranging from one week to one year, one week to six months or one week to one month.

49. The method of any one of claims 44-46 comprising at least 2 subsequent time points.

50. The method of any one of claims 44-47 comprising 2, 3, 4, 5, 6, 7, 8, 9, 10, 11, 12, 13, 14, 15, 16, 17, 18, 19, or 20 subsequent time points.

51. The method of any one of claims 44-48, wherein the treatment is selected from a chemotherapy, immunotherapy, radiation therapy, resection surgery, solid tissue transplant, a stem cell transplant, or a combination thereof.

52. The method of any one of claims 44-48, wherein the treatment is an autologous or allogenic antigen specific T cell therapy.

53. A method of predicting whether a patient is likely to relapse after a remission, comprising:
(a) performing steps (a) through (c) set forth in claim 1; quantifying the magnitude of the immune response from step (c); and comparing the magnitude of the patient's immune response with a reference immune response selected from
(vii) a response generated in a similar method using one or more reference samples from subjects with known treatment outcomes (i.e. known to have relapsed, or not) or
(viii) historical response values from one or more reference samples from subjects with known treatment outcomes (i.e. known to have relapsed, or not);
wherein (A) if the immune response from step (c) is comparable to the reference immune response of a patient known to have relapsed, the patient is determined to have a high likelihood of relapsing; and (B) if the immune response from step (c) is comparable to the reference immune response of a patient known to not have relapsed, the patient is determined to have a low likelihood of relapsing.

54. The method of claim 51, wherein if the patient is determined to have a high likelihood of relapsing, the patient is treated with chemotherapy, immunotherapy, radiation therapy, resection surgery, transplant (solid tissue or stem cell) or a combination thereof.

55. The method of claim 51, wherein if the patient is determined to have a low likelihood of relapsing, steps (a) through (c) set forth in claim 1 are repeated one or more additional times over time to monitor whether any temporal changes in the immune responses elicited by the one or more T cells is detectable.

56. The method of claim 53, wherein if a temporal change in the immune responses elicited by the one or more T cells is detectable, the magnitude of the change is quantified and compared one or more time to the magnitude of the immune response of a reference immune response as set forth in claim 8.

57. The method of claim 54, wherein if the patient is determined to have a high likelihood of relapsing after one or more time of comparing the magnitude of patient's immune response with a reference immune response, the patient is treated with chemotherapy, immunotherapy, radiation therapy, resection surgery, transplant (solid tissue or stem cell) or a combination thereof.

58. A method for detecting changes in the TSA expression profile of a tumor over time comprising:
(a) culturing peripheral blood mononuclear cells (PBMCs) obtained from the subject at a first time point (“baseline”) with a plurality of pepmix libraries, each library comprising a plurality of overlapping peptides that combine to cover a known tumor specific antigen (TSA); wherein culturing results in stimulating the expansion of one or more T cells contained within the PBMCs obtained from the subject;
(b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries;
(c) measuring and quantifying the magnitude of any immune response elicited by the one or more T cells in response to the contacting in (b);
(d) repeating steps (a) through (c) one or more additional times with PBMCs obtained from the subject at one or more subsequent time points;
(e) comparing the magnitude of the immune responses from the first and one or more of each subsequent quantifying step; and
(f) identifying any immune responses that change in magnitude over time; wherein a change in an immune response elicited by a particular pepmix covering a known TSA, or by a peptide comprised in such a pepmix, indicates a change in the expression of that TSA by a tumor in the patient.

59. The method of claim 56, wherein the first time point is before the patient receives a treatment for the cancer and each subsequent time point is after the patient receives the treatment.

60. The method of claim 56 or 57, wherein the first time point and a first subsequent time point are separated by a period ranging from one week to one year, one week to six months or one week to one month.

61. The method of any one of claims 56-58 comprising at least 2 subsequent time points.

62. The method of any one of claims 56-59 comprising 2, 3, 4, 5, 6, 7, 8, 9, 10, 11, 12, 13, 14, 15, 16, 17, 18, 19, or 20 subsequent time points.

63. The method of any one of claims 56-60, wherein the treatment is selected from a chemotherapy, immunotherapy, radiation therapy, resection surgery, solid tissue transplant, a stem cell transplant, or a combination thereof.

64. The method of any one of claims 56-60, wherein the treatment is an autologous or allogenic antigen specific T cell therapy.

65. A method for monitoring the in vivo persistence of antigen specific T cells that have not been genetically modified and that have been administered to a subject, the method comprising
(a) culturing peripheral blood mononuclear cells (PBMCs) obtained from the subject at a first time point (“baseline”) with a plurality of pepmix libraries, each library comprising a plurality of overlapping peptides that combine to cover a known tumor specific antigen (TSA); wherein culturing results in stimulating the expansion of one or more T cells contained within the PBMCs obtained from the subject;
(b) contacting one or more of the T cells that have undergone expansion according to step (a) with (i) at least one of the said pepmix libraries included in the plurality of pepmix libraries, or (ii) at least one of the plurality of overlapping peptides contained in one of said pepmix libraries;
(c) measuring whether the contacting in (b) results in an immune response elicited by the one or more T cells; wherein the measuring optionally comprises quantifying the magnitude of any immune response elicited by the one or more T cells in response to the contacting in (b);
(d) repeating steps (a) through (c) one or more additional times with PBMCs obtained from the subject at one or more subsequent time points;
(e) comparing the baseline immune response with one or more subsequent immune responses, wherein optionally the comparing comprises comparing the magnitude of the immune responses,
(f) wherein the detection of an immune response from one or more of the subsequent time points that is greater than or equal to the immune response generated by T cells obtained from the patient at the baseline time indicates that the antigen specific T cells still persist;
(g) and wherein the detection of an immune response from one or more of the subsequent time points that is less than the immune response generated by T cells obtained from the patient at the baseline time indicates that the antigen specific T cells no longer persist.

66. The method of any one of claims 33-65, wherein the tumor specific antigen is a neoantigen.
